(19) United States
(12) Patent Application Publication
(10) Pub. No.: US 2011/0105788 A1
(43) Pub. Date: May 5, 2011

(54) Polymorphic Form of an Aminoindan Mesylate Derivative

(75) Inventors: Stephen Benedict David Winter, Barcelona (ES); Maria Carmen Burgarolas Montero, Girona (ES)

(73) Assignee: MEDICHEM, S.A., Barcelona (ES)

(21) Appl. No.: 12/934,824

(22) PCT Filed: Mar. 27, 2009
(86) PCT No.: PCT/IB2009/005506

§ 371 Date: Jan. 13, 2011

Related U.S. Application Data
(60) Provisional application No. 61/064,824, filed on Mar. 28, 2008.

(51) Int. Cl. C07C 309/04

(52) U.S. Cl. 562/114

(57) ABSTRACT

The invention relates to a polymorphic form of rasagiline mesylate, and to processes for preparing the same.

### CROSS REFERENCE TO RELATED APPLICATION

This application claims priority to U.S. Provisional Application No. 61/064,824, filed Mar. 28, 2008, which is expressly incorporated herein by reference in its entirety.

### BACKGROUND OF THE INVENTION

1. Field of the invention

The invention relates to a polymorphic form of rasagiline mesylate, and to processes for preparing the same.

2. Background of the invention

Rasagiline mesylate is an active pharmaceutical substance with an empirical formula of C₁₂H₁₃N.CH₄O₃S and a molecular weight of 267.34. Rasagiline mesylate is the international common accepted name for R-(+)-N-propargyl-1-aminoindan mesylate, which is represented in Formula I.

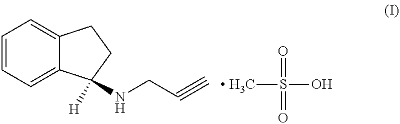

Rasagiline mesylate is an active substance indicated for the treatment of the signs and symptoms of idiopathic Parkinson disease as initial monotherapy and as adjunct therapy to levodopa. Rasagiline is a selective irreversible inhibitor of the B-form of monoamine oxidase enzyme (MAO-B). In the United States, rasagiline mesylate is marketed under the name Azilect™ for the treatment of early Parkinson disease.

It has been reported in the Summary Basis of Approval of the European Medicines Agency (EMEA) for rasagiline mesylate that rasagiline mesylate has no known polymorphs. In this regard, Example 6B of U.S. Pat. No. 5,532,415; E.P. Patent No. 0812190B1 and E.S. Patent No. 2235170T3, disclose the isolation of rasagiline mesylate after crystallization from isopropanol. However, U.S. Pat. No. 5,532,415 only reports the melting point of the solid rasagiline mesylate (157° C.) and a specific rotation value ([ ]_{D}=22° C.), and does not mention the polymorphic form of said compound.

Polymorphism is very common among pharmaceutical substances. It is commonly defined as the ability of any substance to exist in two or more crystalline phases that have different arrangement and/or conformation of the molecules in the crystal lattice. Different polymorphs differ in their physical properties such as melting point, solubility, chemical reactivity, etc. Thus, the particular characteristics of the respective polymorphs can appreciably influence pharmaceutical properties such as dissolution rate and bioavailability.

Due to improved drug formulations, showing, for example, better bioavailability or better stability are consistently sought, there is an ongoing need for new or purer polymorphic forms of existing drug molecules. Therefore, there is a need to provide polymorphic forms of rasagiline mesylate.

### BRIEF DESCRIPTION OF THE DRAWINGS

The accompanying drawings, which are included to provide a further understanding of the invention and are incorporated in and constitute a part of this specification, illustrate embodiments of the invention and together with the description serves to explain the principles of the invention. In the drawings:

FIG. 1 illustrates the X-ray powder diffraction (XRD) of rasagiline mesylate Form I; and

FIG. 2 illustrates the infra-red (IR) spectrum of rasagiline mesylate Form I (taken from example 8).

### DESCRIPTION OF THE INVENTION

The application relates to a polymorphic form of rasagiline mesylate, and to processes for the preparation thereof.

In a first aspect, the application includes a polymorphic form of rasagiline mesylate, referred to herein as Form I rasagiline mesylate, which is characterized as having an X-ray powder diffraction pattern having peaks at approximately 9.0, 13.5, 18.1 and 22.9±0.2 degrees 2θ. In another embodiment, rasagiline mesylate of the application is characterized as having an X-ray powder diffraction pattern having peaks at approximately 4.7, 9.0, 13.5, 18.1, 22.9, and 27.3±0.2 degrees 2θ. In yet another embodiment, rasagiline mesylate of the invention is characterized as having an X-ray powder diffraction pattern having peaks at approximately 4.7, 9.0, 13.5, 14.2, 15.1, 16.2, 16.6, 17.4, 18.1, 21.1, 21.5, 22.1, 22.7, 22.9, 23.9, 24.3, 25.1, 26.1, 26.5, 27.3 and 33.0°±0.2 degrees 2θ.

In another aspect, the rasagiline mesylate Form I of the application is characterized as having an IR spectrum having peaks at approximately 3278.3, 2987.4, 2958.2, 2765.3, 2667.3, 2605.8, 2442.4, 1626.8, 1483.9, 1457.5, 1445.8, 1207.7, 1152.1, 1047.1, 1014.7, 778.1, 751.2, 714.2, 645.6, 557.4, 540.3 and 527.3 cm⁻¹. In another embodiment, rasagiline mesylate of the application is characterized as having an IR spectrum having peaks at approximately 3278.3, 2987.4, 2958.2, 2765.3, 2667.3, 2605.8, 2442.4, 1626.8, 1604.8, 1483.9, 1457.5, 1445.8, 1425.4, 1353.4, 1337.7, 1323.9, 1207.7, 1152.1, 1047.1, 1014.7, 958.5, 944.4, 778.1, 751.2, 714.2, 645.6, 557.4, 540.3 and 527.3 cm⁻¹.

Another aspect of the application includes processes for preparing rasagiline mesylate Form I.

The rasagiline mesylate used above for preparing rasagiline mesylate Form I can be rasagiline mesylate obtained by known methods.

Another feature of the application includes a formulation including rasagiline mesylate obtained according to the processes described herein.

### EXAMPLES

The following examples are for illustrative purposes only and are not intended, nor should they be interpreted to, limit the scope of any invention.

### General Experimental Conditions

X-Ray Powder Diffraction (XRD)

The XRD diffractograms were obtained using a RX SIEMENS D5000 diffractometer with a vertical goniometer, a copper anodic tube, and radiation CuK_{α}, λ=1, 54056 Å.

Infrared Spectra (IR)

Fourier transform IR spectra were acquired on a Thermo Nicolet Nexus spectrometer, and samples were characterized in potassium bromide pellets.

### Examples 1-13

### Preparation of Rasagiline Mesylate form I

General procedure: rasagiline mesylate (100 mg) was suspended in a solvent (1 mL) and heated (in a closed vial) at the temperature indicated in the table for 1 hour. The mixtures were allowed to cool to ambient temperature, stirred for 24 hours at this temperature before evaporation of the solvent (by opening the vial and allowing to evaporate under ambient temperature pressure conditions). The results are summarized in Table 1.

TABLE 1

| Example | Solvent | T (° C.) | XRD Result |
|---|---|---|---|
| 1 | Dichloromethane | 25 | Form I |
| 2 | MTBE | 50 | Form I |
| 3 | THF | 50 | Form I |
| 4 | 2-butanone | 70 | Form I |
| 5 | MIBK | 70 | Form I |
| 6 | 2-propanol | 70 | Form I |
| 7 | n-Butanol | 70 | Form I |
| 8 | ethyl Acetate | 70 | Form I |
| 9 | n-butyl acetate | 70 | Form I |
| 10 | i-propyl acetate | 70 | Form I |
| 11 | acetonitrile | 70 | Form I |
| 12 | Toluene | 70 | Form I |
| 13 | Heptane | 70 | Form I |

### Examples 14-17

### Preparation of Rasagiline Mesylate Form I

General procedure: rasagiline mesylate (100 mg) was suspended in a solvent (1 mL) and heated (in a closed vial) at the temperature indicated in the table for 1 hour, causing dissolution. The mixtures were allowed to cool to ambient temperature, stirred for 24 hours at this temperature at which point the product was still dissolved. The product was isolated by evaporation of the solvent (by opening the vial and allowing to evaporate under ambient temperature pressure conditions). The results are summarized in Table 2.

TABLE 2

| Example | Solvent | T (° C.) | XRD Result |
|---|---|---|---|
| 14 | methanol | 50 | Form I |
| 15 | water | 70 | Form I |
| 16 | dimethylformamide | 70 | Form I |
| 17 | water/ethanol (20-80) | 70 | Form I |

### Examples 18-23

### Preparation of Rasagiline Mesylate Form I: Evaporation Studies

General procedure: rasagiline mesylate (100 mg) was dissolved in a solvent (quantity indicated in table) at ambient temperature. The mixtures were evaporated under vacuum on a rotory evaporator with water bath at 40° C. The results are summarized in Table 3.

TABLE 3

| Example | Solvent | Volume (mL) | XRD Result |
|---|---|---|---|
| 18 | dichloromethane | 3 | Form I |
| 19 | chloroform | 2 | Form I |
| 20 | methanol | 0.5 | Form I |
| 21 | ethanol | 2 | Form I |
| 22 | 2-propanol | 10 | Form I |
| 23 | water | 0.5 | Form I |

### Examples 24-27

### Preparation of Rasagiline Mesylate Form I

General procedure: rasagiline mesylate (100 mg) was suspended in a solvent (volume indicated in table 4) and heated at the temperature indicated in the table until completely dissolved. The mixtures were allowed to cool to ambient temperature, stirred for 24 hours at this temperature at which point the product had precipitated. The product was isolated by filtration and dried under vacuum at 40° C. The results are summarized in Table 4.

TABLE 4

| Example | Solvent | Volume (mL) | T (° C.) | XRD Result |
|---|---|---|---|---|
| 24 | dichloromethane | 2 | reflux | Form I |
| 25 | THF | 10 | reflux | Form I |
| 26 | acetone | 16 | reflux | Form I |
| 27 | 2-butanone | 11 | reflux | Form I |
| 28 | MIBK | 20 | reflux | Form I |
| 29 | 2-propanol | 1 | reflux | Form I |
| 30 | n-butanol | 1 | reflux | Form I |
| 31 | n-butyl acetate | 11 | reflux | Form I |
| 32 | chloroform | 1 | 50° C. | Form I |
| 33 | ethanol | 1 | 70° C. | Form I |

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

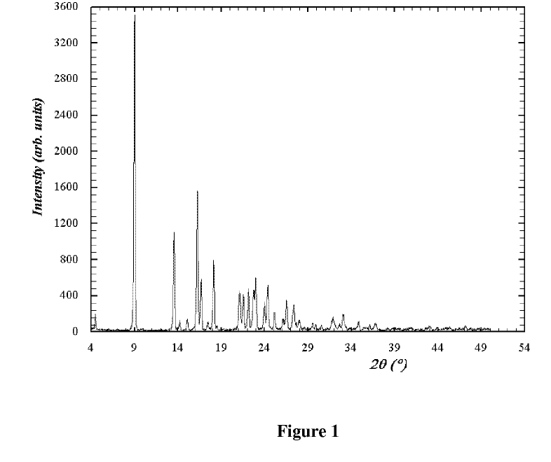

1. A polymorphic form of crystalline R-(+)-N-propargyl-1-aminoindan mesylate, designated Form I rasagiline mesylate, having an X-ray diffraction pattern (2θ) substantially similar to that of FIG. 1.

2. The Form I rasagiline mesylate polymorph of claim 1 having an X-ray powder diffraction pattern having characteristic peaks at approximately 4.7, 9.0, 13.5, 14.2, 15.1, 16.2, 16.6, 17.4, 18.1, 21.1, 21.5, 22.1, 22.7, 22.9, 23.9, 24.3, 25.1, 26.1, 26.5, 27.3 and 33.0°±0.2 degrees 2θ.

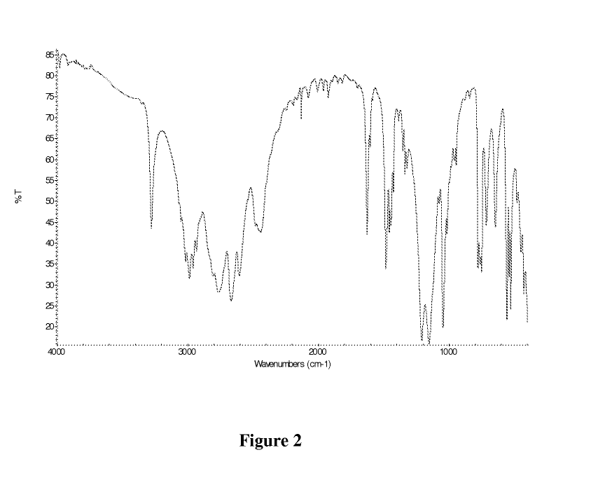

3. A polymorphic form of crystalline R-(+)-N-propargyl-1-aminoindan mesylate, designated Form I rasagiline mesylate, having an IR spectrum substantially similar to that of FIG. 2.

4. The Form I rasagiline mesylate polymorph of claim 3 having an IR spectrum having peaks at approximately 3278.3, 2987.4, 2958.2, 2765.3, 2667.3, 2605.8, 2442.4, 1626.8, 1604.8, 1483.9, 1457.5, 1445.8, 1425.4, 1353.4, 1337.7, 1323.9, 1207.7, 1152.1, 1047.1, 1014.7, 958.5, 944.4, 778.1, 751.2, 714.2, 645.6, 557.4, 540.3 and 527.3 cm⁻¹.
